(19) United States
(12) Patent Application Publication
(10) Pub. No.: US 2001/0055585 A1
(43) Pub. Date: Dec. 27, 2001

(54) FRNK PROTEINS IN THE TREATMENT OF TUMOR CELLS

(76) Inventors: WILLIAM G. CANCE, CARRBORO, NC (US); LI-HUI XU, CARRBORO, NC (US)

Correspondence Address: MYERS BIGEL SIBLEY & SAJOVEC, PO BOX 37428, RALEIGH, NC, 27627, US

(21) Appl. No.: 09/204,134

(22) Filed: Dec. 2, 1998

Related U.S. Application Data
(60) Provisional application No. 60/067,785, filed on Dec. 3, 1997.

(51) Int. Cl. A61K 48/00

(52) U.S. Cl. 424/93.6; 435/320.1; 435/325; 435/69.1; 435/252.3

(57) ABSTRACT

A recombinant virus vector comprising a heterologous nucleic acid segment encoding FRNK protein (including active fragments thereof) operably linked to regulatory sequences directing expression of said FRNK protein in a cell susceptible to infection by said DNA virus vector is disclosed. Also disclosed is isolated mammalian FRNK protein, along with nucleic acids encoding the same and expression cassettes containing such nucleic acids. Methods of using the foregoing to induce the death of tumor cells are disclosed.

### RELATED APPLICATIONS

This application claims priority from Provisional Application Ser. No. 60/067,785, filed Dec. 3, 1997, the disclosure of which is incorporated by reference herein in its entirety.

This work was made with Government support under Grant No. 1 RO1 CA65910 from the National Institutes of Health. The Government has certain rights to this invention.

### FIELD OF THE INVENTION

The present invention concerns the treatment of cancer and cancer cells with FRNK proteins, particularly FRNK proteins expressed from a viral vector. The present invention also concerns human FRNK proteins and DNA encoding the same.

### BACKGROUND OF THE INVENTION

The Focal Adhesion Kinase (FAK) was originally identified in chicken embryo fibroblasts through its physical association with the transforming gene v-Src (M. Schaller et al., Proc. Nat'l Acad. Sci. USA 89: 5192-6 (1992); see also J. Hungerford et al., Journal of Cell Biology, 135:1383-90 (1996)). FAK is an intracellular tyrosine kinase that is localized to focal adhesions, which are the contact points between a cell and its substratum and which serve as anchor points for the cytoskeleton. FAK is the major tyrosine phosphorylated protein in focal adhesions, and is activated by a number of signals, including integrin aggregation (Kornberg, L. J., et al., Proc. Nat'l Acad. Sci. USA 88: 8392-6; (1991); J. Guan et al., Cell Regulation 2: 951-64 (1991) and stimulation by mitogens such as vasopressin, bombesin, endothelin, and bradykinin (L. Leeb-Lundberg et al., Journal of Biological Chemistry 269: 24328-34 (1994); J. Sinnett-Smith, et al., Journal of Biological Chemistry 268(19): 14261-8 (1993); 1. Zachary et al., Journal of Biological Chemistry 267(27): 19031-4 (1992)). Furthermore, FAK forms a complex with paxillin, tensin, and talin, proteins which are part of the cytoskeleton (A. Richardson and T. Parsons, Nature, 380: 538-40 (1996); J. Hildebrand et al., Molecular Biology of the Cell 6: 637-47 (1995); H. Chen et al, Journal of Biological Chemistry, 270(28): 16995-9 (1995)).

The human FAK counterpart was identified in a screen for tyrosine kinases expressed in human high-grade sarcomas (T. Weiner et al., Annals of Surgical Oncology 1: 18-27 (1994)). FAK was subsequently found to be overexpressed at the mRNA level in breast and colon tumors compared to corresponding normal tissues from the same patients (T. Weiner et al., Lancet 342(8878): 1024-5 (1993)). At the protein level, FAK was overexpressed in primary and metastatic tumors, while only minimally detectable levels of FAK expression were found in normal tissue samples from breast, colon, noninvasive soft tissue tumors, (L. Owens et al., Cancer Research, 55: 2752-5 (1995)) and thyroid tumors (L. Owens et al., Annals of Surgical Oncology, 3: 100-5 (1996)). In contrast, elevated expression of other kinases in these tissues was not observed. These findings have been confirmed in other tumor systems (B. Withers et al., Cancer Biochemistry Biophysics 15: 127-39 (1996)).

FAK expression was attenuated by antisense oligonucleotides and it was found that cells lost adherence and underwent apoptosis (L. Xu et al., Cell Growth & Differentiation 7: 413-8 (1996)). However, the utility of antisense oligonucleotides is limited by the potential non-specific inhibition of message stability and other cellular effects. For this reason, there is a need for other potential means of blocking the activity of FAK.

An alternate transcript of the FAK gene, called FRNK, (FAK-related non-kinase) (M. Schaller et al., Molecular & Cellular Biology, 13: 785-91 (1993)), has been described which is encoded by the carboxy-terminal 359 amino acids of FAK. Overexpression of FRNK in chicken embryo fibroblasts inhibits cell spreading on fibronectin, blocks focal adhesion formation, and reduces tyrosine phosphorylation of FAK (A. Richardson. and T. Parsons, supra).

### SUMMARY OF THE INVENTION

We show herein that induction of FRNK expression in tumor cells causes loss of adhesion and cell death. Using an inducible vector, we have demonstrated that FRNK is expressed at the focal adhesions, leading to a rounded morphology of cells and loss of viability. These effects accompanied a decrease in tyrosine phosphorylation of FAK and indicate that FRNK is an inhibitor of FAK in tumor cells and a therapeutic agent for tumors.

A first aspect of the present invention is a recombinant DNA virus vector comprising a heterologous DNA segment encoding FRNK protein operably linked to regulatory sequences directly expression of said FRNK protein in a cell susceptible to infection by said DNA virus vector.

A second aspect of the present invention is isolated mammalian FRNK protein.

A third aspect of the present invention is isolated DNA encoding mammalian FRNK protein.

A fourth aspect of the present invention is an expression cassette comprising an isolated DNA encoding mammalian FRNK protein operably associated with a regulatory segment such as a promoter.

A fifh aspect of the present invention is a method of inducing death in cancer cells, comprising administering to said cells FRNK protein as described above (e.g., by administering a recombinant vector as described above, or by directly administering FRNK protein) in an amount sufficient to kill said cells (e.g., by inducing apoptosis therein).

A sixth aspect of the present invention is a method of treating cancer in a subject in need of such treatment, comprising administering to said subject a treatment effective amount of FRNK protein as described above (e.g., by administering a vector as described above, or by directly administering FRNK protein).

A further aspect of the invention is a method of screening compounds for efficacy in inducing apoptosis in cancer cells, comprising: determining whether said compound specifically binds to FRNK or FAK, the binding of said compound to FRNK or FAK indicating said compound is useful in treating said proliferative disease.

The foregoing and other objects and aspects of the present invention are explained in detail in the drawings herein and the specification set forth below.

### BRIEF DESCRIPTION OF THE DRAWINGS

FIG. 1 (panels A through J) provides the DNA sequence, and the corresponding amino acid sequence in single letter code, of human FRNK protein.

FIG. 2 shows the DNA sequence encoding a FRNK protein useful for carrying out the present invention. A fragment thereof that encodes a protein that induces apoptosis in tumor cells is shown in bold. Numbering is based on (i.e., corresponds to) the numbering of the human focal adhesion kinase (FAK) gene from which this human sequence (sometimes also designated FAK carboxy-terminal domain or “FAK-CD”) was generated.

FIG. 3 shows the DNA sequence of the fragment of the DNA sequence of FIG. 2 shown in bold, and which encodes a protein that induces apoptosis in tumor cells.

FIG. 4 shows the FRNK amino acid sequence derived from human FAK (or human FAK-CD). A fragment thereof that induces apoptosis in tumor cells is shown in bold.

FIG. 5 shows the amino acid sequence of the fragment of FRNK protein shown in bold in FIG. 4, and which induces apoptosis in tumor cells.

### DETAILED DESCRIPTION OF THE INVENTION

Amino acid sequences disclosed herein are presented in the amino to carboxy direction, from left to right. The amino and carboxy groups are not presented in the sequence. Nucleotide sequences are presented herein by single strand only, in the 5′ to 3′ direction.

The production of cloned genes, recombinant DNA, recombinant vectors, proteins and protein fragments by genetic engineering is well known, and can be carried out in accordance with known techniques. See, e.g., U.S. Pat. No. 5,585,269 to Earp et al.; U.S. Pat. No. 5,468,634 to Liu; and U.S. Pat. No. 5,629,407 to Xiong et al. (the disclosures of all United States Patent references cited herein are to be incorporated herein by reference in their entirety.

The term “FRNK protein” as used herein refers to either the alternate transcript of the FAK (M. Schaller et al., supra) or a non-kinase carboxy terminus fragment of FAK (e.g., a FAK fragment having the kinase region deleted, such as the carboxy-terminal 359 amino acids of FAK). In general, FRNK protein retains at least one cytoskeletal binding element of FAK and/or other biological function of FAK (e.g., p130^{cas}SH3 binding, GRAF SH3 binding, GRB SH2 binding, Talin binding, and Paxillin binding (particularly talin binding)). Thus the term “FRNK protein” as used herein thus also includes fragments of the 359 amino acid carboxy terminus of FAK that retain at least one binding function or biological function of FRNK as set forth in Table 3 below, and/or are at least 100, 150, or 200 amino acids in length. Such fragments may be formed by deleting an amino terminus segment from FRNK, by deleting a carboxy terminus segment from FRNK, by deleting an intervening segment from FRNK, and combinations thereof. It will also be appreciated that one or more minor heterologous amino acid segments (e.g., 1-10 amino acids in length) can be inserted into FRNK or FRNK fragments, at the amino terminus, at the carboxy terminus, in an intervening position, or combinations thereof, without changing the function of FRNK. FRNK protein or DNA can be of natural origin from any suitable species, including avian (e.g., chicken) and mammalian (e.g., human).

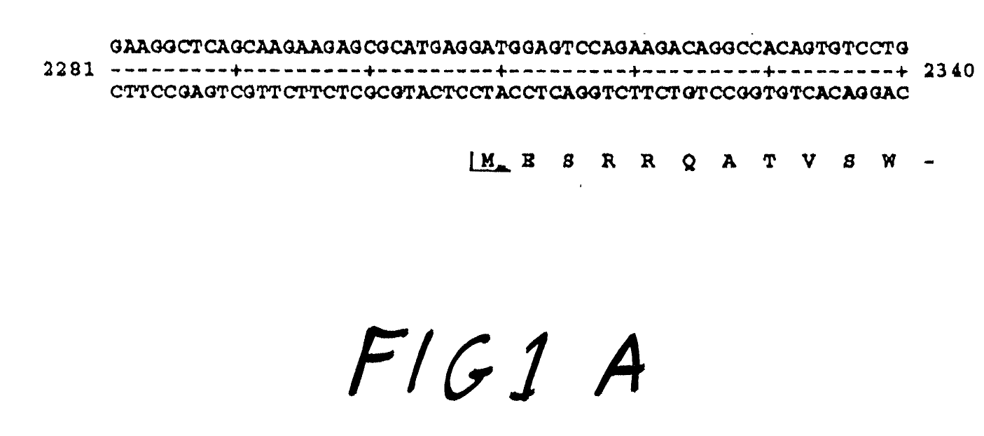

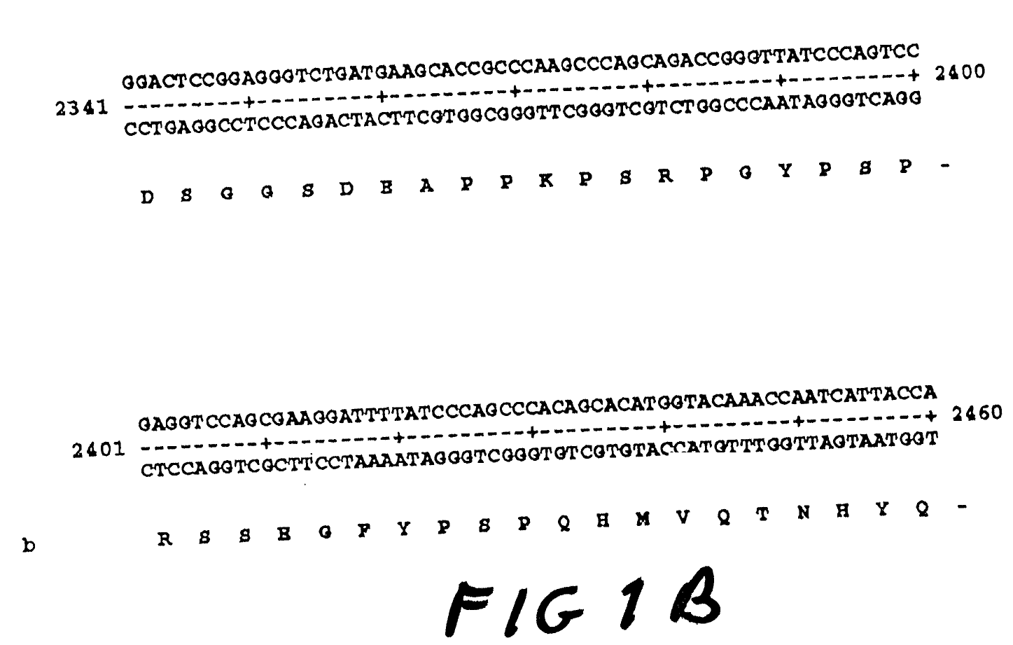

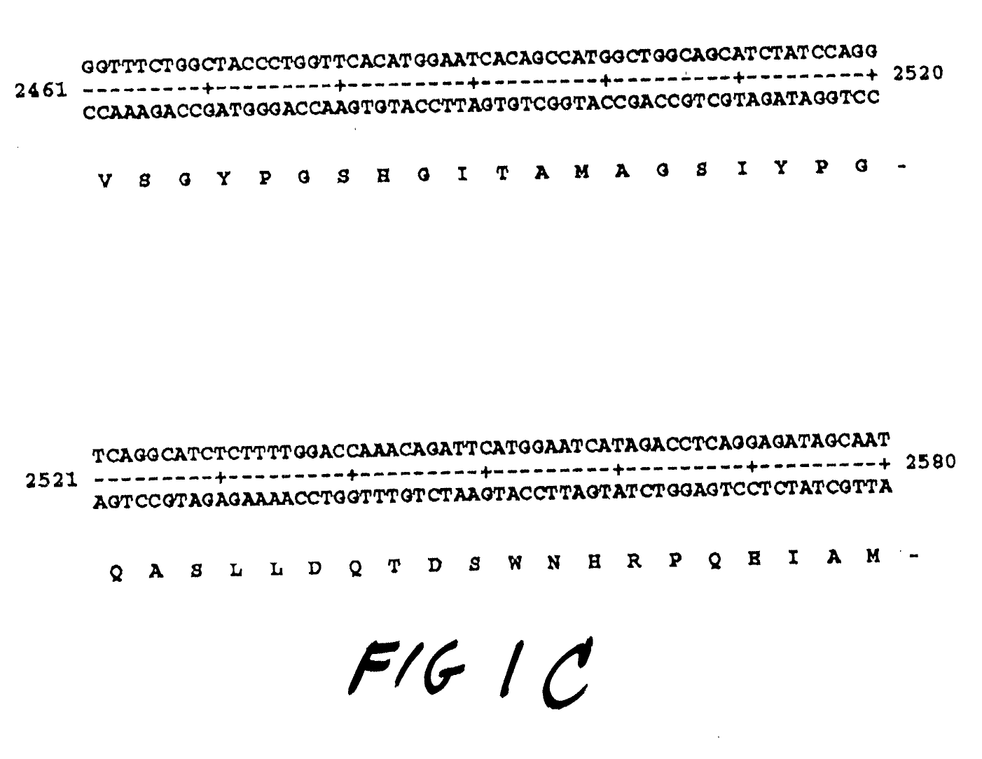

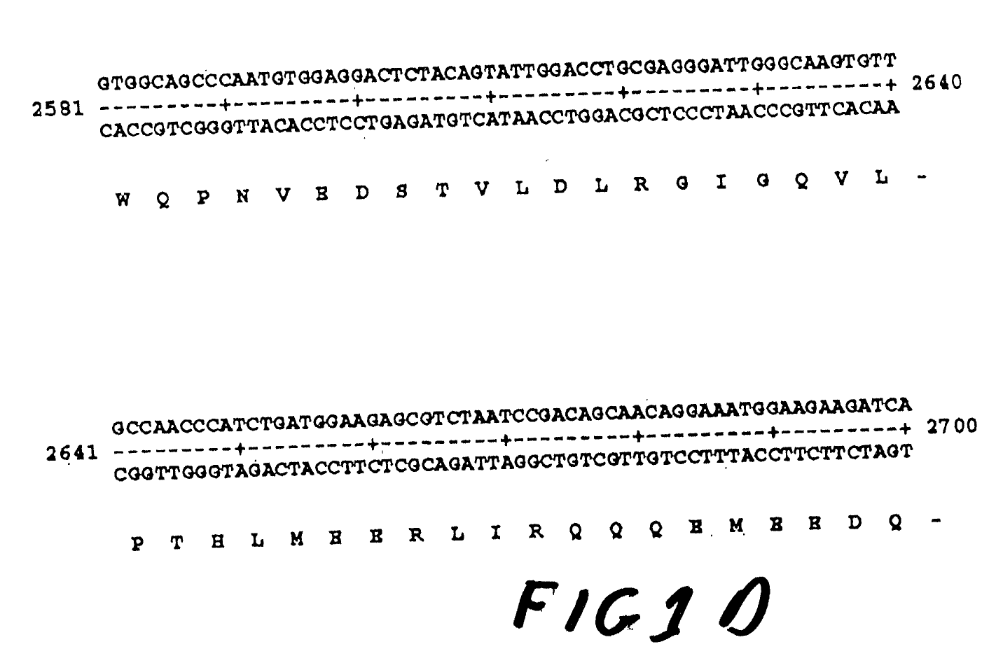

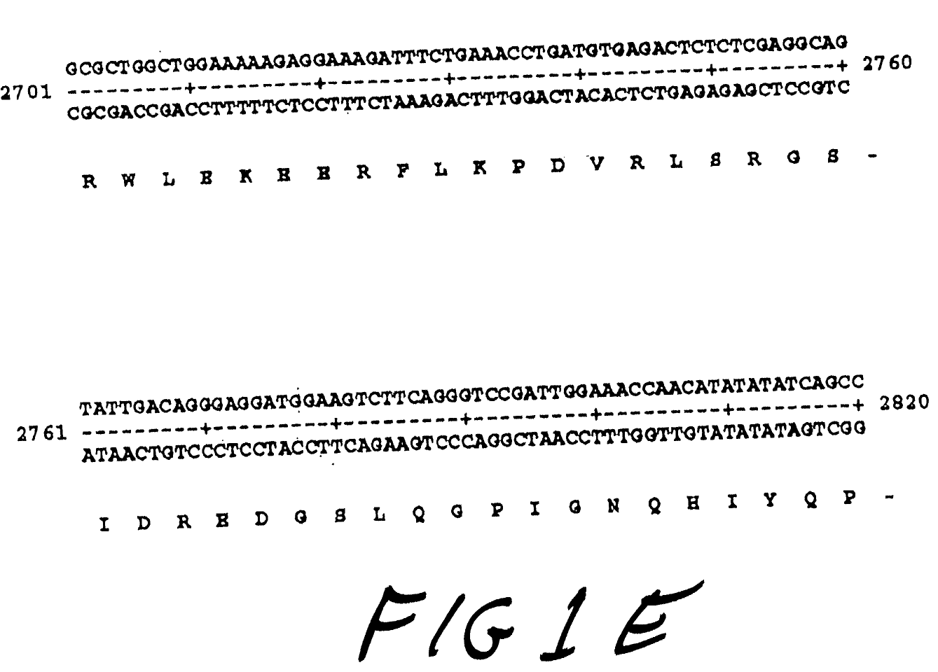

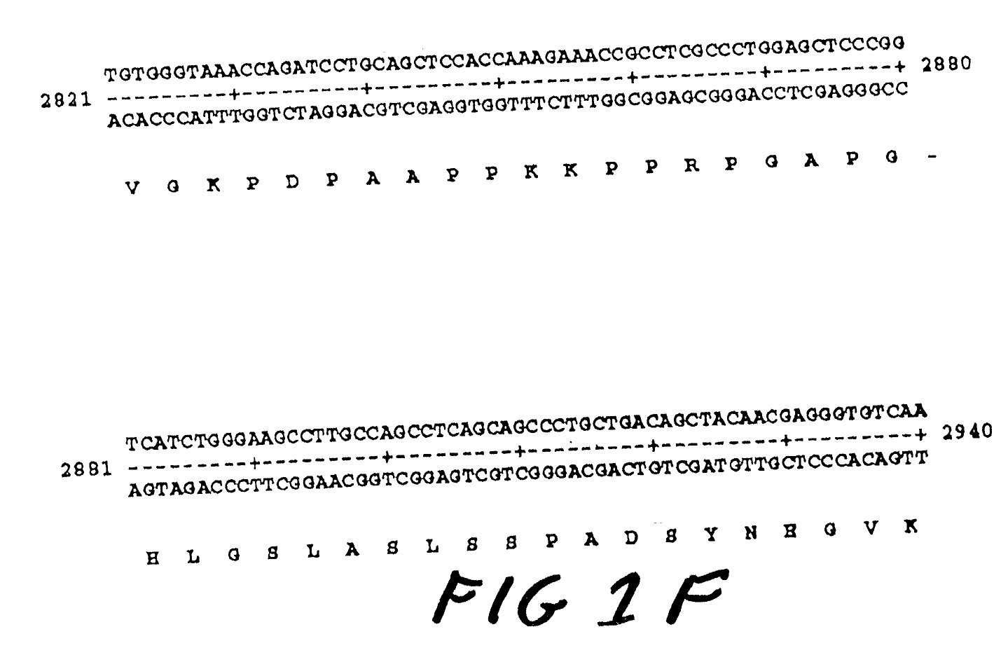

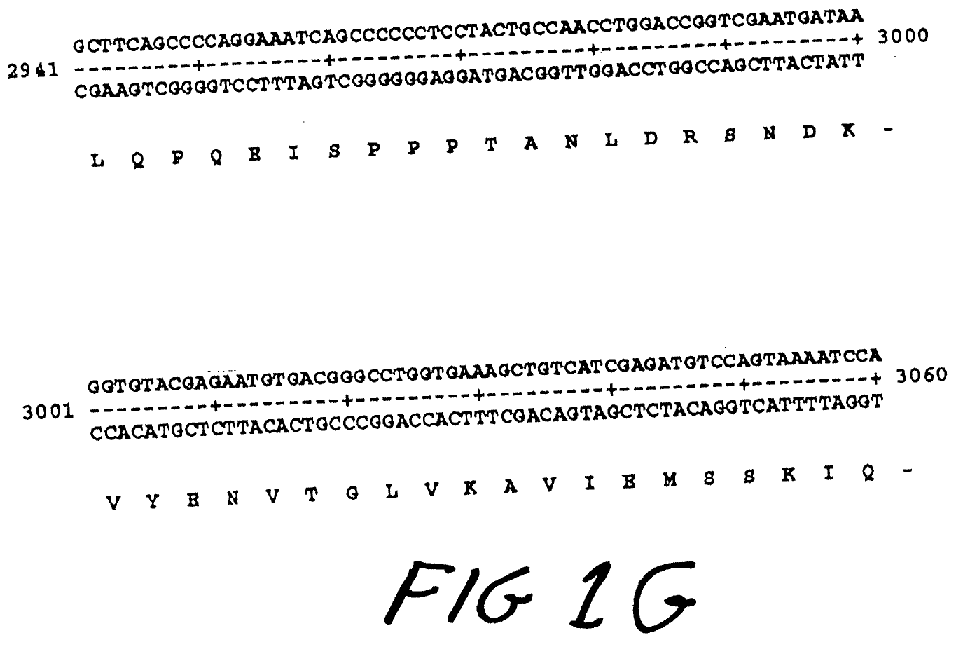

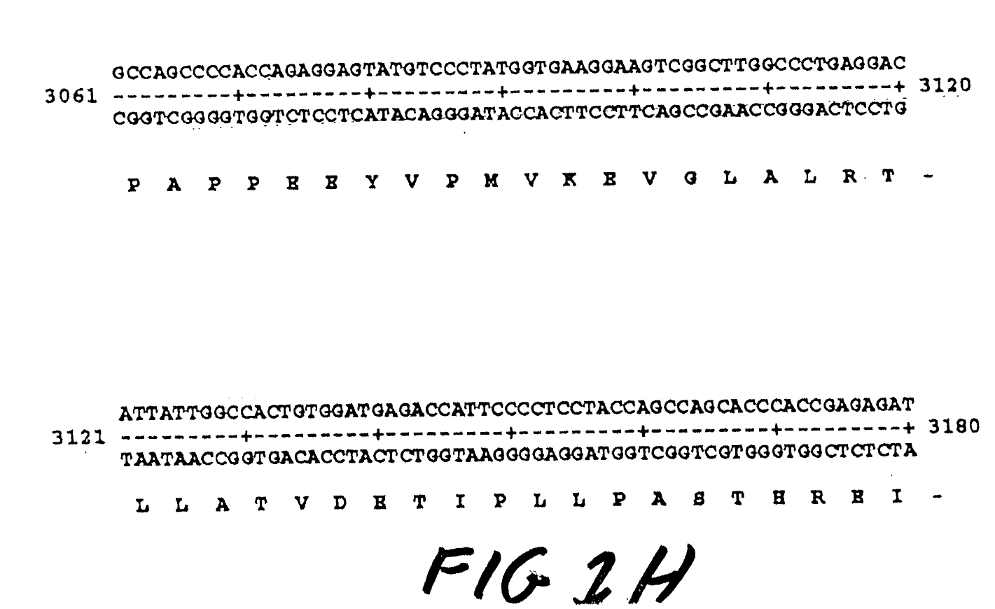

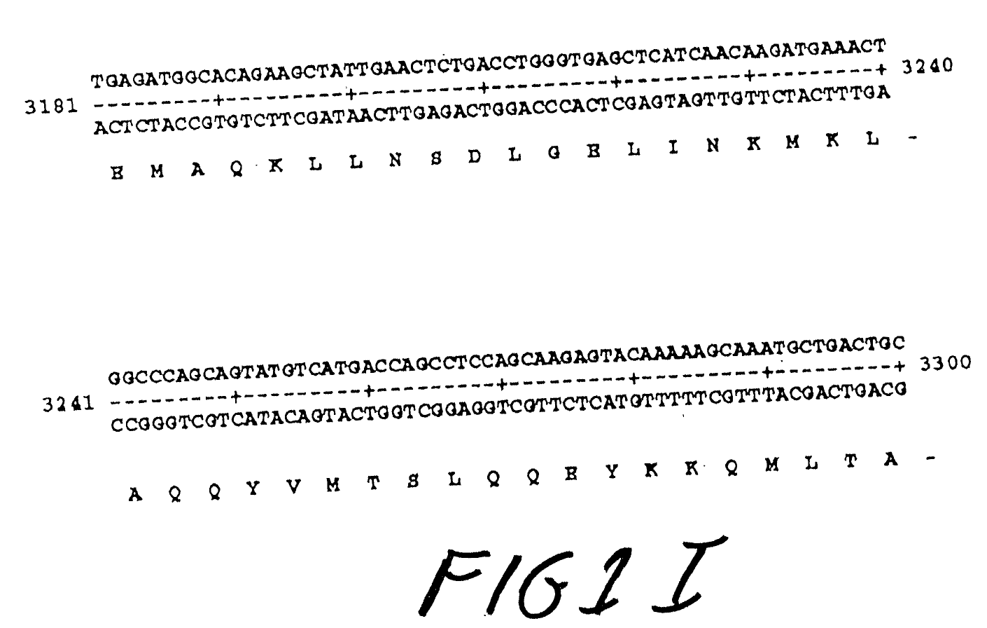

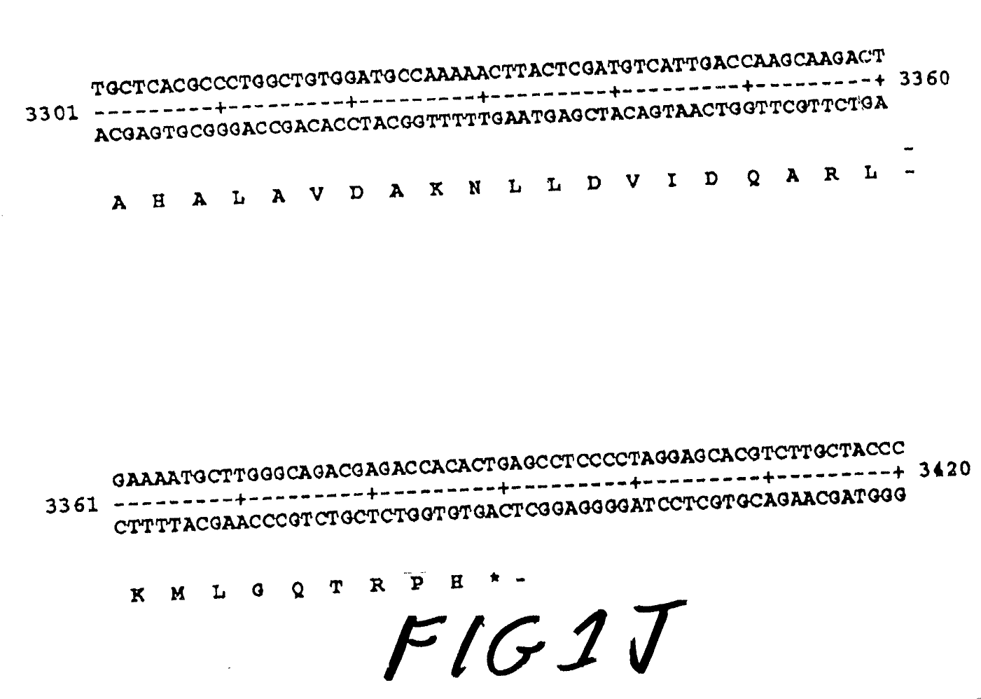

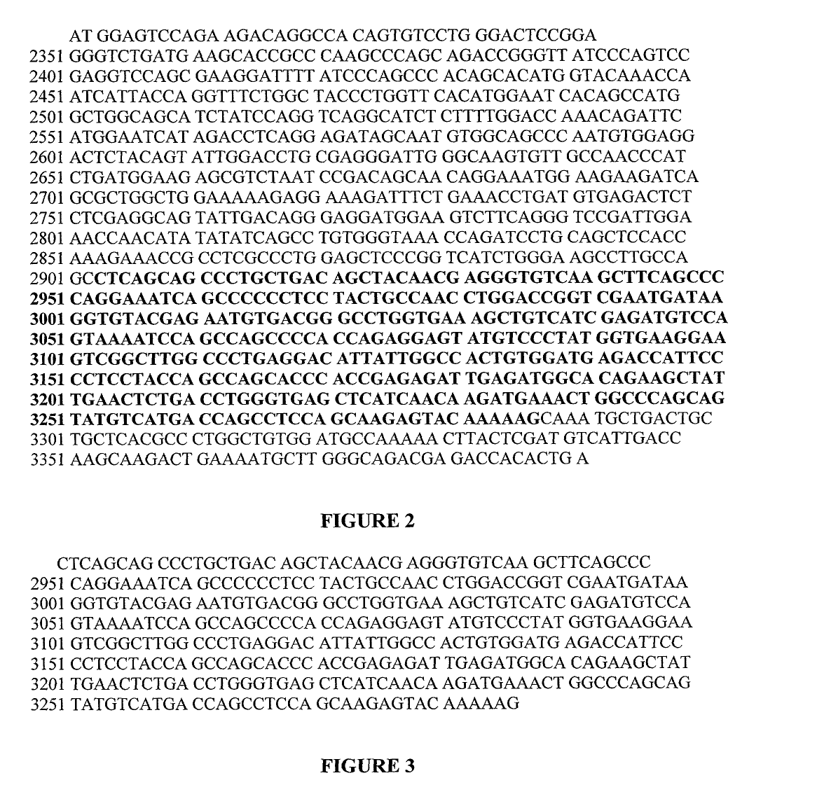

DNA encoding FRNK may be that according to FIG. 1 herein (that is, the coding segment for the carboxy-terminus 359 amino acids of human FAK), or may be taken from the carboxy-terminus of other human FAK transcripts such as that disclosed in G. Whitney et al., DNA and Cell Biology 12, 823-830 (1993). Thus, DNAs from other natural sources (such as other avian or mammalian species) that hybridize to DNA encoding FRNK protein (such as that given in FIG. 1, FIG. 2, or FIG. 3 herein or known DNA encoding chicken FRNK), and which code on expression for a FRNK protein as defined herein (including active fragments thereof), are also an aspect of this invention. Conditions which will permit other DNAs which code on expression for a FRNK protein to hybridize to DNA encoding FRNK as described herein can be determined in accordance with known techniques. For example, hybridization of such sequences may be carried out under conditions of reduced stringency, medium stringency, or even stringent conditions (e.g., conditions represented by a wash stringency of 35-40% Formamide with 5× Denhardt's solution, 0.5% SDS and lx SSPE at 37° C.; conditions represented by a wash stringency of 40-45% Formamide with 5× Denhardt's solution, 0.5% SDS, and lx SSPE at 42° C.; and conditions represented by a wash stringency of 50% Formamide with 5× Denhardt's solution, 0.5% SDS and Ix SSPE at 42° C., respectively, to DNA encoding the FRNK protein disclosed herein in a standard hybridization assay. See J. Sambrook et al., Molecular Cloning, A Laboratory Manual (2d Ed. 1989). In general, sequences which code for FRNK protein will be at least 75% homologous, 85% homologous, or even 90% homologous or more with the sequence encoding FRNK disclosed herein, and encode a protein that retain the biological activity of FRNK as given above. Further, DNAs which code for FRNK proteins as described above, but which differ in codon sequence from these due to the degeneracy of the genetic code, yet code for the same protein, are also an aspect of this invention and can be used, along with the encoded protein, to carry out the present invention.

FIG. 2 shows the DNA sequence encoding a FRNK protein useful for carrying out the present invention. A fragment thereof that encodes a protein that induces apoptosis in tumor cells is shown in bold, and is also shown in FIG. 3. Other nucleic acids that hybridize to these DNAs as described above, and optionally bearing the sequence similarity described above, can also be used to carry out the present invention.

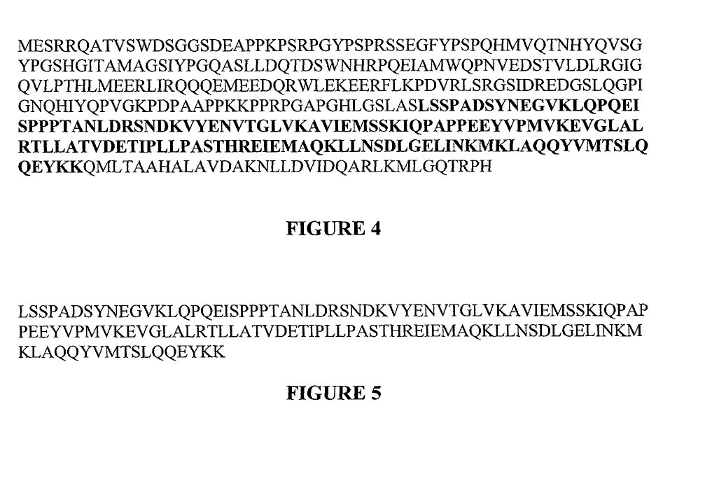

FIG. 4 shows the amino acid sequence, in single letter code, of a FRNK protein useful for carrying out the present invention. A fragment thereof that induces apoptosis in tumor cells is shown in bold, and is also shown in FIG. 5. Other nucleic acids that code on expression for these proteins can also be used to carry out the present invention.

Additional examples of FRNK fragments useful for carrying out the present invention include fragments of the protein encoded by the DNA of FIG. 3 or the protein of FIG. 5. These fragments correspond to positions 891 to 1018 of human FAK. Examples of such fragments include all continuous fragments of these proteins that are 30, 40, 50, or 60 amino acids or more in length. Fragments that incorporate positions 891 or 900 to 940 or 950, or fragments that incorporate the talin binding domain (positions 965-1012) are preferred.

Vectors used to carry out the present invention are, in general, DNA virus vectors, such as papovavirus vectors (e.g., SV40 vectors and polyoma vectors), adenovirus vectors and adeno-associated virus vectors. See generally T. Friedmann, Science 244, 1275 16 (June 1989). RNA virus vectors may also be employed, including but not limited to alphavirus vectors and lentivirus vectors. Examples of lentivirus vectors that may be used to carry out the present invention include but are not limited to Moloney Murine Leukemia Virus vectors, such as those described in U.S. Pat. No. 5,707,865 to Kohn. Examples of alphavirus vectors that may be used include, but are not limited to, those described in U.S. Pat. No. 5,792,462 to Johnston.

Any adenovirus vector can be used to carry out the present invention. See, e.g., U.S. Pat. No. 5,518,913, U.S. Pat. No. 5,670,488, U.S. Pat. No. 5,589,377; U.S. Pat. No. 5,616,326; U.S. Pat. No. 5,436,146; and U.S. Pat. No. 5,585,362. The adenovirus can be modified to alter or broaden the natural tropism thereof, as described in S. Woo, Adenovirus redirected, Nature Biotechnology 14, 1538 (Nov. 1996).

Any adeno-associated virus vector (or AAV vector) can also be used to carry out the present invention. See, e.g., U.S. Pat. No. 5,681,731; U.S. Pat. No. 5,677,158; U.S. Pat. No. 5,658,776; U.S. Pat. No. 5,622,856; U.S. Pat. No. 5,604,090; U.S. Pat. No. 5,589,377; U.S. Pat. No. 5,587,308; U.S. Pat. No. 5,474,935; U.S. Pat. No. 5,436,146; U.S. Pat. No. 5,354,678; U.S. Pat. No. 5,252,479; U.S. Pat. No. 5,173,414; U.S. Pat. No. 5,139,941; and U.S. Pat. No. 4,797,368.

The regulatory sequences, or the transcriptional and translational control sequences, in the vectors can be of any suitable source, so long as they effect transcription and translation of FRNK in the target cells. For example, commonly used promoters are the LacZ promoter, and promoters derived from polyoma, Adenovirus 2, and Simian virus 40 (SV40). See, e.g., U.S. Pat. No. 4,599,308.

Once prepared, the recombinant vector can be reproduced by (a) propagating the vector in a cell culture, the cell culture comprising cells that permit the growth and reproduction of the vector therein; and then (b) collecting the recombinant vector from the cell culture, all in accordance with known techniques. The viral vectors collected from the culture may be separated from the culture medium in accordance with known techniques, and combined with a suitable pharmaceutical carrier for administration to a subject. Such pharmaceutical carriers include, but are not limited to, sterile pyrogen-free water or sterile pyrogen-free saline solution. If desired, the vectors may be packaged in liposomes for administration, in accordance with known techniques.

FRNK protein may be produced by inserting an expression cassette that expresses FRNK protein in a suitable expression vector and transforming host cells therewith in a culture medium, isolating the FRNK protein from the host cells, and purifying the FRNK protein as desired, all in accordance with known techniques.

FRNK protein may be used in essentially the same manner as the FRNK vectors described herein, and may be combined in a pharmaceutical carrier essentially as described above, or may be combined with a non-sterile carrier such as water or a buffer solution for use as a laboratory reagent as discussed below.

The present invention can be used to treat a variety of different types of cancer and tumors, particularly malignant (and preferably solid) tumors of epithelial or mesenchymal cells. Examples of cancers that can be treated by the present invention include breast cancer, melanoma, lung cancer, colon cancer, leukemia (a liquid or non-solid tumor), soft tissue and bone sarcomas, neuroendocrine tumors such as islet cell carcinoma or medullary carcinoma of the thyroid, squamous carcinomas (particularly of the head and neck), adenocarcinomas, etc. The treatment of breast cancer is a particularly preferred target for carrying out the present invention. The term “treat” as used herein refers to any type of treatment that imparts a clinical improvement in the condition of the patient, or delays the progression of the disease.

While the present invention is primarily concerned with the treatment of human subjects, the invention may also be carried out on animal subjects such as dogs, cats, and horses for veterinary purposes.

Any suitable route of administration can be used to carry out the present invention, depending upon the particular condition being treated. Suitable routes include, but are not limited to, intraveneous, intrarterial, intrathecal, intraperitoneal, intramuscular, and intralesional injection. Intralesional injection is currently preferred.

For administration of FRNK protein, the dosage will depend upon factors such as the particular disorder, the formulation, the condition of the patient, the route of administration, etc., and can be optimized for specific situation. In general, the dosage is 0.01, 0.05 or 0.1 up to 20, 40 or 60 mg/Kg subject body weight The dosage of the recombinant vector administered will depend upon factors such as the particular disorder, the particular vector chosen, the formulation of the vector, the condition of the patient, the route of administration, etc., and can be optimized for specific situations. In general, the dosage is from about 10⁷, 10⁸, or 10⁹ to about 10¹¹, 10¹², or 10¹³ plaque forming units (pfu).

In addition to their pharmaceutical or veterinary use, the proteins and recombinant vectors of the present invention (sometimes also referred to as “active agents” herein) are useful in vitro to induce a loss of adhesion in susceptible cells in culture, to distinguish cells in culture based on their response to the active agents, to induce apoptosis, etc. Such techniques are useful for both carrying out cell culture procedures and for experimental purposes.

FRNK protein may also be administered to susceptible solid tumors, such as tumors of muscle tissue or muscle tissue origin, by direct injection of an expression cassette encoding and expressing FRNK protein into the tumor as so-called “naked” DNA, in the manner described in U.S. Pat. No. 5,589,466.

Antibodies that specifically (i.e., selectively) bind to FRNK protein as described above are a further aspect of the invention. In general, such antibodies do not specifically bind to FAK. Such antibodies are useful, among other things, for the affinity purification of FRNK of for the detection of FRNK expression in vitro in accordance with standard techniques. Such antibodies include polyclonal and monoclonal antibodies, antibody fragments, humanized or chimeric antibodies, etc. that retain the combining region that specifically binds to FRNK protein. The antibodies may be of any type of immunoglobulin, including but not limited to IgG and IgM immunoglobulins. The antibodies may be of any suitable origin, such as chicken, goat, rabbit, horse, etc., but are preferably mammalian. When used for detecting FRNK expression the antibodies may be labelled by conjugating them, directly or indirectly, to a suitable detectable group (e.g., an enzyme, a radioisotope, a detectable protein such as green fluorescent protein, etc.) in accordance with known techniques.

As also noted above, the present invention also provides a method of screening a compound for efficacy in the inducing apoptosis, inhibiting the growth, and/or treating hyperproliferative or cancer cells. The methods can be carried out in a cell or cells, or can be carried out in essentially cell free preparation. The method may be carried out as a single assay, or may be implemented in the form of a high throughput screen in accordance with a variety of known techniques such as phage display, yeast two-hybrid systems, etc. In one embodiment, the method of screening compounds comprises determining (e.g., in vitro) whether said compound specifically binds to FRNK as described above, or to focal adhesion kinase (FAK) (preferably mammalian, and most preferably human, FAK). The determining step can be carried out by screening for binding of a test compound or probe molecule to the entire full length FRNK or FAK, or to a peptide fragment thereof as described above. The binding of the compound to the FRNK or FAK indicates that the compound is useful in the treatments described above. Such techniques can be carried out by contacting a probe compound to the FRNK or FAK or fragment thereof in any of the variety of known combinatorial chemistry techniques (including but not limited to split pool techniques, chip-based techniques and pin-based techniques). Any suitable solid support can be used to imobilize the FRNK, FAK, or a fragment thereof to find specific binding partners thereto (or immobilize the members of the library against which the FRNK, FAK or fragment thereof is contacted to find specific binding partners thereto), and numerous different solid supports are well known to those skilled in the art. Examples of suitable materials from which the solid support may be formed include cellulose, pore-glass, silica gel, polystyrene, particularly polystyrene cross-linked with divinylbenzene, grafted copolymers such as polyethyleneglycol/polystyrene, polyacrylamide, latex, dimethylacrylamide, particularly cross-linked with N,N′bis-acrylolyl ethylene diamine and comprising N-t-butoxycarbonyl-beta-alanyl-N′acrylolyl hexamethylene diamine, composites such as glass coated with a hydrophobic polymer such as cross-linked polystyrene or a fluorinated ethylene polymer to which is grafted linear polystyrene, and the like. Thus the term “solid support” includes materials conventionally considered to be semi-solid supports. General reviews of useful solid supports that include a covalently-linked reactive functionality may be found in Atherton et al., Prospectives in Peptide Chemistry, Karger, 101-117 (1981); Amamath et al., Chem. Rev. 77: 183 (1977); and Fridkin, The Peptides, Vol. 2, Chapter 3, Academic Press, Inc., pp 333-363 (1979). The solid support may take any suitable form, such as a bead or microparticle, a tube, a plate, a microtiter plate well, a glass microscope cover slip, etc.

The present invention can be used with probe molecules, or libraries (where groups of different probe molecules are employed), of any type. In general, such probe molecules are organic compounds, including but not limited to that may be used to carry out the present include oligomers, non-oligomers, or combinations thereof. Non-oligomers include a wide variety of organic molecules, such as heterocyclics, aromatics, alicyclics, aliphatics and combinations thereof, comprising steroids, antibiotics, enzyme inhibitors, ligands, hormones, drugs, alkaloids, opioids, benzodiazepenes, terpenes, prophyrins, toxins, catalysts, as well as combinations thereof. Oligomers include peptides (that is, oligopeptides) and proteins, oligonucleotides (the term oligonucleotide also referred to simply as “nucleotide” herein) such as DNA and RNA, oligosaccharides, polylipids, polyesters, polyamides, polyurethanes, polyureas, polyethers, poly (phosphorus derivatives) such as phosphates, phosphonates, phosphoramides, phosphonamides, phosphites, phosphinamides, etc., poly (sulfur derivatives) such as sulfones, sulfonates, sulfites, sulfonamides, sulfenamides, etc., where for the phosphorous and sulfur derivatives the indicated heteroatom for the most part will be bonded to C, H, N, O or S, and combinations thereof. Numerous methods of synthesizing or applying such probe molecules on solid supports (where the probe molecule may be either covalently or non-covalently bound to the solid support) are known, and such probe molecules can be made in accordance with procedures known to those skilled in the art. See, e.g., U.S. Pat. No. 5,565,324 to Still et al., U.S. Pat. No. 5,284,514 to Ellman et al., U.S. Pat. No. 5,445,934 to Fodor et al. (the disclosures of all United States patents cited herein are to be incorporated herein by reference in their entirety).

Test compounds used to carry out the present invention may be of any type, including both oligomers or non-oligomers of the types described above in connection with probe molecules above. Again, such test compounds are known and can be prepared in accordance with known techniques.

Where multiple different probe molecules are desired to be tested, a screening substrate useful for the high throughput screening of molecular interactions, such as in “chip-based” and “pin-based” combinatorial chemistry techniques, can be prepared in accordance with known techniques. All can be prepared in accordance with known techniques. See, e.g., U.S. Pat. No. 5,445,934 to Fodor et al., U.S. Pat. No. 5,288,514 to Ellman, and U.S. Pat. No. 5,624,711 to Sundberg et al.

In the alternative, screening of libraries of probe molecules may be carried out with mixtures of solid supports as used in “split-pool” combinatorial chemistry techniques. Such mixtures can be prepared in accordance with procedures known in the art, and tag components can be added to the discreet solid supports in accordance with procedures known in the art. See, e.g., U.S. Pat. No. 5,565,324 to Still et al.

The present invention is explained in greater detail in the following non-limiting Examples.

### EXAMPLE 1

### Expression of FRNK in Tumor Cells

This example demonstrates that the exogenous expression of FRNK leads to loss of adhesion and loss of viability in human breast cancer and human melanoma cells. Materials, methods and results are shown below.

A. Materials and Methods

Cell lines and Cell Culture Conditions.

The BT 474 human breast ductal carcinoma cells, NIH 3T3 mouse fibroblasts and Cos-7 monkey kidney cell line were purchased from ATCC (Rockville, Md.). The C8161 human melanoma cell line was kindly provided by Dr. Bernard E. Weissman.

Vector Construction.

1. FAK vector construction: A FAK cDNA clone was isolated from the HT29 human colon cancer cell line (Sturge and Cance, unpublished data), and was sub-cloned into the pcDNA3 (Invitrogen) expression vector, incorporating an in-frame sequence for the expression of the hemaglutanin epitope (HA) containing amino acids of YPYDVPDYA at the amino terminus of the protein.

2. FRNK vector construction.

A. pCRII-FRNK: HA-tagged FRNK was amplified by PCR with Taq DNA polymerase using pBluescript-FAK cDNA as a template. The primers were as follows: 5′-CGGGGTACCGTCGACGCCGCCACCATGGACTACCCCTATGATGTGCCC GATTACGCTGAGTCCAGAAGACAGGCC and 5′-ATTAACCCTCACTAAAG. PCR-amplified HA-FRNK was cloned into pCRII vector (Invitrogen).

B. pLX-FRNK: pCRII-FRNK was digested with XhoI and subsequently cloned into the pLXSN expression vector (Miller, A. D. and G. J. Rosman, Biotechniques, 1989. 7(9): p. 980-2, 984-6, 989-90). pLX-FRNK or pLXSN vector was transfected into RD, BT474, C8161, and NIH 3T3 cells and assayed for growth inhibition.

C. pSAR-MT-FRNK. pCRII-FRNK was digested with KpnI and cloned into pBluescript SK vector digested with the same restriction enzyme, and then subcloned into BamHi site of pSAR-MT vector (Kindly provided by Dr. Bert Vogelstein) containing a zinc inducible metallothionein promoter.

D. pCMV4-FRNK: pCRII-FRNK was digested with KpnI and then cloned into the Kpnl site of pCMV4 expression vector, which contains a CMV promoter.

Growth Inhibition Assay.

pLXFRNK or pLXSN vector was transfected into BT474, C8161, and NIH 3T3 cells and assayed for its growth inhibitory activity. Briefly, 10 μg of pLXFRNK construct or pLXSN vector was mixed with 8 μg/ml lipofectamine (Life Technologies, Inc.) in serum-free media, left for 30 minutes, and added to cells and incubated for 6-8 hours at 37° C. Cells were grown in media containing 10% FBS for 48 hours at 37° C. 5×10⁵ cells were then plated in complete media containing 500 μg/ml Geneticin (G418) (Life Technologies, Inc.) for BT474 and NIH3T3 cells and 100 μg/ml for C8161 cells. After two weeks of selection in G418, cells were fixed with 3:1 methyl alcohol: acetic acid and stained with 1% crystal violet. Percentage of growth inhibition was determined based on the number of colonies in each plate.

Induction of FRNK Expression.

10 μg pSAR-MT-FRNK or pSAR-MT vector and 1 μg neo-resistant vector pSV2neo were transfected into BT474 and C8161 cells, and selected with G418 as described above. Ten colonies from each cell line were isolated, expanded and tested for the ability to induce FRNK expression with ZnSO₄. The concentrations of ZnSO₄ for induction of FRNK expression were 75 μM and 50 μM for BT474 and C8161 cells, respectively. Subclones which expressed FRNK following treatment with ZnSO₄ were expanded for further analysis.

Replating Assay.

For the replating assay, suspended cells were harvested and stained with trypan blue. 1×10⁵ trypan-blue excluding cells were replated into 6-well plates in complete medium as described above and incubated for 24 hours at 37° C. Adherent cells were trypsinized and counted, and percentage of replated FRNK-expressing cells was normalized by that of replated vector control cells.

Immunofluorescence.

BT474 or C8161cells containing the pSAR-MT-FRNK or pSAR-MT plasmids were plated into 4-well chamber slides (Nalge Nunc International) and incubated overnight at 37° C. Cells were then treated with 75 μM or 50 μM ZnSO₄ for 24 hours, fixed with 3.7% formaldehyde for 15 minutes, and permeabilized with 0.1% Triton X-100 for two minutes on ice. Cells were stained with anti-HA monoclonal antibody (HA11, BAbCO) for one hour at room temperature, followed by goat anti-mouse IgG conjugated with rhodamine. Cells were washed with PBS, and positive cells were visualized and photographed with a Zeiss fluorescence microscope.

Transfection of Cos-7 Cells.

Subconfluent Cos-7 cells were cotransfected with 8 μg of pCMV4-FRNK and 2 μg of pcDNA3-FAK mixed with 20 μl of lipofectarnine (Gibco BRL) in serum-free DMEM and incubated overnight at 37° C., the cells were grown in DMEM medium containing 10% FBS for 24 hours at 37° C. Cells were then lysed in NP-40 lysis buffer and analyzed for FAK expression, tyrosine phosphorylation and kinase activity as described below.

Western Blotting, Immunoprecipitations and Kinase Assay.

For the preparation of whole-cell lyastes, cells were lysed in NP-40 lysis buffer recipe as described (Cance, W. G., et al., Cell Growth & Differentiation, 1994. 5(12): p. 1347-55), and 50 μg of cell lysate was analyzed for FRNK expression by Western blotting. Membranes were immunoblotted with anti-HA monoclonal antibody (12CA5, Boehringer Mannheim, Indianapolis, Ind.). For immunoprecipitation, 250 μg of cell lysate was incubated with 1 μg of anti-FAK polyclonal antibody (C20, Santa Cruz) or 10 μg of anti-HA monoclonal antibody (12CA5) in the presence of protein A/G-agarose (Calbiochem). The beads were washed three times with NP-40 buffer, then boiled for 3 minutes in SDS-PAGE loading buffer, and the precipitated proteins were analyzed by Western blot. Membranes were probed with anti-phosphotyrosine monoclonal antibody (4G10, Upstate Biotech.) or anti-FAK antibody (C20, Santa Cruz). Proteins were visualized using the ECL detection system (Amersham).

For FAK kinase assay, anti-HA immunoprecipitates from transfected COS-7 cells were washed three times with NP-40 buffer, then washed twice with Tyrosine Kinase Buffer (10 mM HEPES, pH 7.4 and 5 mM MnCl₂) and resuspended in 25 μl of the same buffer. Kinase reactions were initiated with 10 μCi of [γ³²P]-ATP, incubated at 30° C. for 15 min., and stopped with SDS-PAGE sample loading buffer and analyzed by SDS-PAGE. Following electrophoresis, the gel was dried and autoradiographed.

B. Results

1. FRNK Inhibits Growth of Tumor Cells.

In chicken embryo fibroblasts, FRNK has been identified as an alternate transcript of FAK, consisting of 537 nucleotides of unique 5′ sequence spliced to the 3′ 2234 nucleotides of the avian FAK cDNA, and the FRNK protein encoded by this transcript appears to be an inhibitor of FAK. The human counterpart of FRNK has not been cloned, although we have identified a 41 kD protein that co-migrates with chicken FRNK using an antibody to the carboxy terminus of FAK (data not shown). The sequences surrounding the initiating methionine of the avian FRNK sequence are identical to the human FAK sequence, so we expressed the carboxy-terminal 359 amino acids of FAK and determined its effects on tumor cell growth. Although the precise identity and sequence of the human FRNK protein have not been determined, we will refer to these 359 amino acids of FAK as “FRNK” for simplicity. To distinguish this exogenous FRNK sequence from the endogenous FAK protein, FRNK was cloned in frame with the Hemagglutanin (HA) epitope-tag sequence at the amino terminus into the pLXSN vector. The pLXSN-FRNK plasmid was subsequently transfected into BT474 human breast cancer cells and C8161 human melanoma cells and grown under neomycin selection. In each of these tumor cell lines, expression of FRNK caused inhibition of colony formation compared to pLXSN vector control (Table 1). Colonies that emerged following two weeks of G418 selection did not express detectable levels of FRNK (data not shown). In contrast, the growth of NIH 3T3 mouse fibroblasts was not as strongly inhibited by FRNK as the tumor cell lines (Table 1).

TABLE 1
Expression of FRNK inhibits cell growth.

Colony formation (%)^{a}

| Cell line | Vector | FRNK |
|---|---|---|
| BT474 | 100 ± 0 | 22.5 ± 2.3 |
| C8161 | 100 ± 0 | 11.0 ± 11.6 |
| NIH 3T3 | 100 ± 0 | 71.5 ± 1.7 |

^{a}5 × 10⁵ cells were plated in selective media after transfected with FRNK or vector. After two weeks of selection, cells were fixed with 3:1 methyl alcohol:acetic acid and stained with 1% crystal violet. Percentage of growth inhibition was determined based on the number of colonies in FRNK transfected cells vs that of vector transfected cells.
^{b}Standard deviation for triple independent experiments.

2. Induction of FRNK Expression Causes a Loss of Adhesion and Tumor Cell Death.

Because FRNK inhibited colony formation in tumor cells, we wished to further study the nature of this growth inhibition. HA-FRNK was cloned into a metallothionein-inducible expression vector, pSAR-MT (Morin, P. J., B. Vogelstein, and K. W. Kinzler, Proceedings of the National Academy of Sciences of the United States of America, 1996. 93(15): p. 7950-4.), and transfected into BT474 and C8161 cells. After G418 selection, stable clones of BT474 and C8161 cells transfected with pSAR-MT-FRNK were isolated and evaluated for inducible FRNK expression. We found three clones from BT474 and one clone from C8161 that expressed FRNK after 24 hours of zinc induction. In each cell line, FRNK expression reached the highest levels at 10 and 24 hours, then decreased at 48 hours following zinc addition (data not shown). In contrast, cells transfected with vector alone did not express detectable levels of FRNK after zinc treatment (data not shown).

Next, we sought to determine whether FRNK induction caused a phenotypic change in these tumor cells. Cells were treated in the presence or absence of zinc and analyzed by immunofluorescence with anti-HA antibody. After 6 hours of induction, the FRNK protein had localized to the focal adhesions (data not shown). After 24 hours, it appeared that 10-15% of both the BT474 and C8161 cells expressed FRNK, with the immunostaining demonstrating diffuse localization throughout the cell (data not shown). FRNK was minimally detectable in uninduced cells, and was not expressed in cells containing the pSAR-MT plasmid (data not shown). Most notably, the cells that expressed detectable levels of FRNK demonstrated a distinct, rounded morphology that lead to their detachment from their substratum (data not shown). In contrast, the cells that did not express FRNK remained adherent to their culture plates. Furthermore, these rounded cells were not found in the ZnSO₄-treated, vector control cells, indicating that this morphology was not due to the zinc treatment. Thus, these results suggested that FRNK localized to the focal adhesion contacts following induction, and that this expression ultimately lead to a rounded morphology and loss of adhesion.

We next determined whether the cells that lost adherence upon FRNK induction still remained viable. Equal numbers of cells in suspension following zinc treatment from either the vector control or the FRNK-expressing cells were re-plated into 6-well plates and allowed to adhere and grow for 24 hours. In the vector control cell populations, 42% of BT474 and 60% of C8161 cells gained adherence 24 hours after replating, suggesting that there was a reversible loss of adherence from the zinc treatment itself, possibly reflecting the ability of zinc to interfere with ion-bound adhesion molecules (data not shown). In contrast, only 14% of the FRNK-expressing BT474 and C8161 cells re-adhered (data not shown), suggesting that the presence of FRNK in these cells caused an irreversible loss of adherence and consequent loss of viability. We analyzed these FRNK-expressing non-adherent cells by flow cytometry and TUNEL assays to determine whether they had undergone apoptosis. By both assays, a small fraction of apoptotic cells were detected (data not shown), suggesting the possibility of either apoptotic or non-apoptotic mechanisms of cellular death.

3. FRNK Interrupts FAK Function in Tumor Cells.

We analyzed the level of FAK phosphorylation following FRNK induction by immunoprecipitating FAK and blotting for total phosphotyrosine content. After 24 hours of FRNK induction in BT474 cells, the tyrosine phosphorylation of FAK was decreased (data not shown), while the levels of p125^{FAK} remained unchanged, demonstrating that induction of FRNK resulted in decreased p125^{FAK} activity. To exclude the possibility that this decrease in tyrosine phosphorylation was a secondary effect of the cells' loss of viability, we transiently co-expressed both FAK and FRNK in Cos-7 cells. In these experiments we found that FRNK reduced both the tyrosine phosphorylation of p125^{FAK} as well as its autophosphorylation activity (data not shown).

### EXAMPLE 2

### Adenovirus Expression of FRNK in Tumor Cells

This Example demonstrates the exogenous expression of FRNK in tumor cells by a recombinant adenovirus containing FRNK cDNA. Materials, methods and results are shown below.

A. Materials and Methods

Construction and Preparation of Recombinant Adenovirus Containing FRNK.

HA-tagged FRNK was cloned into pACCMV.PLPASR(+) vector, and cotransfected with 10 μg of Adenovirus type5 dl309 DNA, digested with XbaI and ClaI, in 293 cells using CaPO₄, in accordance with known techniques. After transfection, cells were overlaid with 2% low melt agarose containing complete media, then fed every 3 days. After about 11 days, plaques had appeared. These were picked using pasteur pipets, and “inoculated” into 0.5 ml media. 100 ul of this media was used to amplify the plaques by infecting confluent 293 cells in 6 well plates. Presence of FRNK cDNA in AdFRNK was confirmed by PCR using primer against FRNK. Adenoviruses carrying FRNK were propagated in 293 cells, purified by two cesium chloride density centrifugation, titered by plaque assay, and expressed in plaque forming units (pfu) (Graham and Prevec, Methods in Molecular Biology 7, 109-128 (1991)). Adenovirus carrying the β-galactosidase gene (β-gal) served as a control.

Adenovirus Vector Infection of Cells In Vitro.

Infection Efficiency.

Cells were plated at 5×10⁴ per well in six-well culture plates and allowed to attach for 24 hours, and then nonadherent cells were removed by gentle washing with PBS. Cells were then infected with AdLacZ at the multiplicity of infection (MOI) of 1000, 500, 250, 125, 62.5 and 31.25 pfu/ml for 24 hours, cells were then washed with PBS, fixed with 2% formaldehyde, 0.2% glutaraldehyde for 5 minutes on ice, and stained for β-gal activity. The efficiency of gene transfer using the adenoviral vector was determined by X-Gal staining.

Percentage of Detached Cells After AdFRNK Infection.

To quantitate the percentage of detached cells following Ad vector infection, cells were plated at 2×10⁶ per 100 mm dished, allowed to attach for 24 hours, and then exposed to AdFRNK or AdLacZ for 24 hours at different MOI for each cell line (Table 2). At 24 hours after Ad vector infection the detached cells floating in the culture medium were harvested by centrifugation at 1000 rpm for 5 minutes. The remaining cells attached to the dishes were harvested using typsin-EDTA solution. The number of detached and adherent cells were counted using a hemocytometer and the remaining cells were then lysed in NP-40 lysis buffer. FRNK expression in these cells were analyzed by Western blotting using anti-HA antibody.

Detection of Apoptosis After AdFRNK Infection in BT474 Human Breast Cancer Cells.

Detection of apoptotic cells by fluorescense microscopy was performed by Tunel assay using the ApopTag-kit (Oncor). Briefly, detached cells were harvested by centrifugation, washed one time with PBS, fixed with 3.7% formaldehyde for 10 minutes on ice, and then resuspended in 80% ethanol. Cells were then smeared onto glass slides, air dried, fixed with 80% ethanol and permeabilized with 0.1% Triton X-100 for 2 minutes on ice. Cells were washed with PBS and incubated with terminal deoxynucleotide transferase for one hour at 37° C. followed by anti-digoxigenin-fluorescein for 30 minutes at room temperature. Cells were washed with PBS and positive cells were visualized with a Zeiss fluorescence microscope.

To confirm this, DNA was also extracted to demonstrate the DNA fragmentation. Briefly, detached cells infected with AdFRNK or attached cells from AdLacZ using trypsin-EDTA were harvested by centrifugation, lsyed in lysis buffer (lmM EDTA, 50 mM Tris,pH 8.0, 0.5% sarcosyl), and treated with 0.5 mg/ml proteinase K at 56° C. overnight, and then treated with 0.5 mg/ml RNAse A for additional 2 hours at 56° C. DNA was then extracted three time with phenol-chloroform, one time with chloroform, ethanol precipitated. 1 mg of DNA was run on 1.2% agarose gel at 70 volts for 1.5 hours, stained with 0.5 μg/ml ethidium bromide, examined under UV light and photographed.

B. Results

Infection with AdFRNK Causes Tumor Cells to Lose Adhesion.

Human tumor cells, including BT474, C8161, RD (above) and HT29 colon carcinoma cells lost adhesion at a different degree 24 hours following infection with AdFRNK (Table 2), while there was no loss of adhesion in AdLAcZ control cells. In contrast, FRNK did not cause a significant loss of adhesion in normal human cells, including HaCaT normal kerotinocytes, HMEC mammary epithilial cells and normal rat smooth muscle cells (data not shown).

TABLE 2
Infection with adenovirus-FRNK in vitro.

| | | FRNK | % suspended | Apoptosis | |
|---|---|---|---|---|---|
| Cell type | MOI | expression | cells | Tunel¹ | Ladder² |
| BT474 | 40 | positive | >95% (n = 4) | positive | positive |
| C8161 | 80 | positive | 60% (n = 3) | NA | NA |
| RD | 160 | positive | 33% (n = 1) | NA | NA |
| HT29 | 50 | positive | 10% (n = 2) | NA | NA |
| HaCaT | 50 | positive | 4% (n = 2) | NA | NA |
| HMEC | 100 | analyzing | 1% (n = 1) | NA | NA |

¹Tunel assay Apo Tag-HT
²Ethidium Bromide-stained agarose gel of DNA extracted from FRNK infected BT474

AdFRNK causes BT474 Cells to Undergo Apoptosis.

Among all the cells infected with AdFRNK, the most significant biological effects were observed in BT474 cells. In these cells, FRNK expression was detected as early as 4 hours, and reached higher levels at 8, 16 and 24 hours after viral infection (data not shown). Most significantly, greater than 95% of cells became suspended 16 hours after infection, and most of these cells died by apoptosis characterized by DNA fragmentation (data not shown). In contrast, there was no loss of adhesion in AdLacZ control cells. AdFRNK caused loss of adhesion in several different tumor cell lines at a different degree, however, whether FRNK causes apoptosis in these cells are underway to be analyzed.

### EXAMPLE 3

### FRNK Deletion Mutants

To further define the region(s) of FRNK which are responsible for the growth inhibitory effects, we made a series of deletion mutants of human FRNK based on the known binding sites to other molecules. The segment deleted for each deletion mutant is given in Table 3 (deleted sequences refers to the nucleotide sequences deleted, as numbered according to the numbers assigned to the corresponding full-length human FRK sequence). FRNK of FRNK mutants were transfected into several different cell lines and assayed for growth inhibition. The results are shown in Table 4.

TABLE 3
FRNK deletion Mutants.

| | | SITE IN | SEQUENCE | FUNCTION |
|---|---|---|---|---|
| CON- | DELETED | FAK/ | IN | IN |
| STRUCT | SEQUENCES | FRNK | FAK/FRNK | FAK/FRNK |
| FRNK ΔC1 | 700-733 | Proline | EAPPKPSR | p130^{cas} SH3 |
| | | 712/715 | | binding |
| FRNK ΔC2 | 734-852 | | | ? |
| FRNK ΔC3 | 853-891 | Proline | APPKKPPRP | GRAF SH3 |
| | | 876 | | binding |
| FRNK ΔC4 | 891-965 | Tyrosine | YENV | GRB2 SH2 |
| | | 925 | | binding |
| FRNK ΔC5 | 965-1012 | | FAT sequence | Talin |
| | | | | binding |
| FRNK | 853-1012 | | FAT sequence | Paxillin |
| ΔC3-5 | | | | binding |
| FRNK ΔC6 | 1018-1042 | | | ? |

TABLE 4
Growth inhibitory effect of FRNK and FRNK mutants (%).

| | | | ΔC1 | ΔC2 | ΔC4 | ΔC5 | ΔC3-5 |
|---|---|---|---|---|---|---|---|
| | | | (700- | (734- | (891- | (965- | (853- |
| Cells | pLXSN | FRNK | 733) | 852) | 965) | 1012) | 1012) |
| RD | 0 | 70 | | | | | 88 |
| | 0 | 50 | | | | | 39 |
| | 0 | 37 | | | | 71 | 56 |
| | 0 | 55 | 67 | | 83 | 55 | 47 |
| | 0 | 63 | 71 | | 87 | 93 | 77 |
| C8161 | 0 | 97 | | | | 98 | 67 |
| | 0 | 76 | 97 | 99 | 98 | 98 | 48 |
| | 0 | 95 | 95 | | 94 | 81 | 19 |
| Meljuso | 0 | 70 | | | | | 79 |
| | 0 | 31 | | | | 62 | 78 |
| MDA231 | 0 | 60 | | | | | 89 |
| | 0 | 62 | | | | 71 | 43 |
| BT474 | 0 | 75 | | | | | 88 |
| | 0 | 80 | | | | | 70 |
| | 0 | 78 | | | | | |
| 3T3 | 0 | 30 | | | | | 55 |
| | 0 | 52 | 22 | 21 | 76 | 72 | 36 |
| | 0 | 27 | | | | | |

these data indicate that numerous different FRNK deletion mutants are also active in accordance with the present invention. The DNA sequence encoding a particularly preferred FRNK fragment is given in FIG. 3 above, and the corresponding amino acid sequence (in single letter code) is given in FIG. 5 above.

### EXAMPLE 4

### Normal Mammary Cells are Resistant to Transduction of FRNK

Normal human mammary epithelial cells (HMEC) were infected with AdFRNK at a MOI (multiplicity of infection) of 40 for 24 hours. A high level of FRNK expression was observed by Western blot using the anti-HA antibody. Although all the cells expressed FRNK protein, they remained adherent and showed no detectable apoptosis. The cells did exhibit some increased cell-cell contacts, and appeared to have a more fibroblastic cytoskeletal appearance than vector control-infected cells. Immunofluorescence using the anti-HA antibody demonstrated tyat FAK-CD localized to focal adhesions, pl25FAK was displaced from the focal adhesions, but paxillin and vinculin remained in focal adhesions (data not shown). Furthermore, FRNK protein was not degraded following displacement from the focal adhesions as demonstrated by Western blot using the anti-FAK carboxy terminal antibody. These observations further confirm that FAK is an excellent target for tumor therapeutics, and that normal cells appear to have a resistance to the effects of FAK inhibitors.

### EXAMPLE 5

### Transduction of FRNK in Other Breast Cell Lines

The effect of FRNK in other cell lines was tested to determine whether the effect noted above was cell line dependent. MCF7 breast cancer cells were transduced with FRNK in essentially the same manner described above, and approximately 90% of the cells became detached from the dishes and underwent apoptosis. In contrast, when MCF10A normal breast epithelial cells were transduced with AdFRNK for 24 hours, there was no significant loss of adhesion and no detectable apoptosis. This provides additional evidence of the “therapeutic window” of FRNK sensitivity between normal and transformed cells.

The foregoing is illustrative of the present invention, and is not to be construed as limiting thereof. The invention is defined by the following claims, with equivalents of the claims to be included therein.

We claim:

1. A recombinant DNA virus vector comprising a heterologous DNA segment encoding FRNK protein operably linked to regulatory sequences directling expression of said FRNK protein in a cell susceptible to infection by said DNA virus vector.

2. A recombinant vector according to claim 1, wherein said DNA virus vector is selected from the group consisting of adenovirus and adeno-associated virus vectors.

3. A recombinant vector according to claim 1, wherein said DNA virus vector is an adenovirus vector.

4. A recombinant vector according to claim 1, wherein said heterologous DNA segment encodes a FRNK protein selected from the group consisting of avian FRNK protein and mammalian FRNK protein.

5. A recombinant vector according to claim 1 in a pharmaceutically acceptable carrier.

6. A method of making a recombinant vector, comprising:
(a) propagating a recombinant vector according to claim 1 in a cell culture, said cell culture comprising cells that permit the growth and reproduction of said recombinant vector therein; and then
(b) collecting said recombinant vector from said cell culture.

7. Isolated mammalian FRNK protein.

8. An isolated DNA encoding mammalian FRNK protein.

9. An expression cassette comprising an isolated DNA according to claim 8 operably associated with a promoter.

10. A method of inducing apoptosis in cancer cells, comprising administering to said cells FRNK protein or an active fragment thereof an amount sufficient to induce apoptosis therein.

11. A method according to claim 10, wherein said cancer cells are selected from the group consisting of breast cancer cells, lung cancer cells, colon cancer cells, and melanoma cells.

12. A method according to claim 10, wherein said cancer cells are breast cancer cells.

13. A method according to claim 10, wherein said administering step is carried out by administering to said cells a recombinant DNA virus vector comprising a heterologous DNA segment encoding FRNK protein operably linked to regulatory sequences directing expression of said FRNK protein in said cells.

14. A method of treating cancer in a subject in need of such treatment, comprising administering to said subject a treatment effective amount of FRNK protein or an active fragment thereof.

15. A method according to claim 14, wherein said cancer is selected from the group consisting of breast cancer, lung cancer, colon cancer, and melanoma.

16. A method according to claim 14, wherein said cancer is breast cancer.

17. A method according to claim 14, wherein said administering step is carried out by administering to said patient a recombinant DNA virus vector comprising a heterologous DNA segment encoding FRNK protein operably linked to regulatory sequences directing expression of said FRNK protein in said patient.

18. A method of screening compounds for efficacy in inducing apoptosis in cancer cells, comprising:
determining whether said compound specifically binds to FRNK or FAK;
the binding of said compound to FRNK or FAK indicating said compound is useful in treating said proliferative disease.

19. A method according to claim 18, wherein said determining step is carried out by split pool combinatorial chemistry.

20. A method according to claim 18, wherein said determining step is carried out by chip-based combinatorial chemistry.

21. A method according to claim 18, wherein said determining step is carried out by pin-based combinatorial chemistry.

22. A method according to claim 18, wherein said determining step is carried out by phage display.

23. A method according to claim 18, wherein said compound is an oligomeric compound.

24. A method according to claim 18, wherein said compound is a non-oligomeric compound.
